(19) United States
(12) Patent Application Publication
(10) Pub. No.: US 2010/0090129 A1
(43) Pub. Date: Apr. 15, 2010

(54) APPARATUS AND METHOD FOR THE PREVENTION OF INFESTATION INSIDE A VOLUME OF INTEREST

(76) Inventor: Avi Klayman, Tel-Aviv (IL)

Correspondence Address: DANIEL J SWIRSKY, 55 REUVEN ST., BEIT SHEMESH, 99544, IL

(21) Appl. No.: 12/447,944

(22) PCT Filed: Oct. 29, 2007
(86) PCT No.: PCT/IL07/01311

§ 371 Date: Dec. 28, 2009

(30) Foreign Application Priority Data
Oct. 30, 2006 (IL) 178936

(51) Int. Cl. G21K 5/00

(52) U.S. Cl. 250/492.1

(57) ABSTRACT

The present invention discloses apparatus [100] for the prevention of infestations inside a volume of interest (VOI) caused by pathogens. The apparatus comprising a first vent [110] supplying a fluid into said VOI; a chamber [140] supplying a fluid to said first vent; a second vent [120] supplying a fluid into said chamber; and, a radiation emitter [130] emitting radiation into said chamber hence disabling said pathogens. The invention also presents a method of preventing of infestations inside a volume of interest caused by pathogens. This method comprising steps of supplying fluid into said volume of interest through a first vent; supplying fluid to said first vent through a chamber; supplying fluid into said chamber through a second vent; and, emitting an effective dosage of radiation into said chamber and hence disabling said pathogens. The invention further teaches a method for the prevention of infestations caused by pathogens inside a volume of interest. This method comprises steps of flowing fluid from a chamber into said volume of interest; flowing fluid into said chamber from outside of said volume of interest; and, emitting an effective dosage of radiation into said chamber and hence disabling said pathogens.

### FIELD OF THE INVENTION

The present invention generally relates to apparatus and method for the prevention of infestation, infection, or disease. More specifically, the present invention relates to apparatus and method for the prevention of infestation inside a volume of interest.

### BACKGROUND OF THE INVENTION

It is common for one biological organism to infest another, for example herbivorous insects may infest plants, or bacteria may infest animals, or human beings may be infected with viruses. It is often desirable to prevent such infestation, for example to prevent insects from infesting crop plants, or to prevent bacteria from infecting domesticate animals, or to prevent disease from human beings. Toxic chemicals (pesticides) are often employed to kill infecting organisms or to slow their multiplication. The adverse effects of pesticides are well known, and are described, for example, in U.S. Pat. No. 5,974,728, which discloses a method and apparatus for the non-toxic control of insects and weeds. U.S. Pat. No. 5,974,728 suggests actively and mechanically separating the infesting organism from the infested, and heating the first while they are separated from the last. However, this step of separation is impossible in many cases, for example in the case of bacterial infestation, or it may harm the infested organism. Another patent application, WO8809616, describes a method for protection of cultivated plants, which employs microwave radiation, but does not separate the infested from the infesting, and therefore may harm both. Both patents suggest deploying means of disinfection in close proximity of the infected, which is often more expensive and time consuming than employing static means that can serve the same purpose at a distance. Furthermore, the expense of deploying means of disinfection in close proximity of each and every possibly infected organism implies periodic disinfection or removal of infestation rather than the prevention of infection or infestation.

A cost-effective apparatus or method for the prevention of infestation, without resorting to harmful chemicals, without the need to separate the infested from the infesting, and without the requirement of deploying equipment in close proximity to the infested, thus meets a long felt need.

### SUMMARY OF THE INVENTION

Thus the present invention provides apparatus and method for the prevention of infestation inside a volume of interest, without resorting to harmful chemicals, without any step of separating the infested from the infesting, and without a requirement of deploying any equipment in close proximity to the infested.

It is an object of the present invention to provide apparatus for the prevention of infestations caused by pathogens inside a volume of interest comprising a first vent supplying fluid (such as air) to the volume of interest, a chamber supplying fluid to the first vent, a second vent supplying fluid to this chamber, and a radiation emitter emitting radiation into said chamber a radiation emitter emitting radiation into the chamber hence disabling said pathogens.

It is in the scope of the present invention to provide apparatus as defined above, wherein the chamber comprises any of the following: a baffle, a filter, a lure or bait.

It is also in the scope of the present invention to provide apparatus as defined above, also comprising a detector of any of the following properties: the fluid flow, the fluid composition, or the emitted radiation.

It is also in the scope of the present invention to provide apparatus as defined above, wherein the radiation comprises electromagnetic radiation.

It is also in the scope of the present invention to provide apparatus as defined above, wherein the electromagnetic radiation comprises any of the following: microwave radiation, infrared radiation, ultraviolet radiation, visible light, gamma radiation, laser radiation, maser radiation.

It is also in the scope of the present invention to provide apparatus as defined above, wherein the electromagnetic radiation is characterized by relatively strong emission in any of the following frequency ranges, in 1/cm units, selected inter alia from a group consisting of about 3300 to about 3500; about 250 to about 3300; and about 150 to about 250.

It is also in the scope of the present invention to provide apparatus as defined above, wherein the radiation comprises ultrasonic waves.

It is also an object of the present invention to provide a method for preventing infestations inside a volume of interest by supplying fluid (such as air) to the volume of interest through a first vent, supplying fluid to the first vent through an fluid flow chamber, supplying fluid to the chamber through a second vent, and emitting radiating into the chamber; and

It is also an object of the present invention to provide a method for the preventing infestations inside a volume of interest by moving fluid into the volume of interest from a fluid flow chamber, moving fluid into the chamber from outside the volume of interest, and emitting radiation into the chamber.

It is in the scope of the present invention to provide a method as defined above, comprising either baffling or filtering the fluid flow in the chamber, or both.

It is also in the scope of the present invention to provide a method as defined above, comprising attracting the infesting organisms to enter the chamber.

It is also in the scope of the present invention to provide a method as defined above, comprising controlling the radiation emitted into the chamber.

It is also in the scope of the present invention to provide a method as defined above, comprising any of detecting properties of radiation, detecting properties of the flowing fluid, or detecting the rate of fluid flow.

It is also in the scope of the present invention to provide a method as defined above, wherein emitting radiation comprises emitting electromagnetic waves, especially any of the following: microwave radiation, infrared radiation, ultraviolet radiation, visible light, gamma radiation, laser radiation, maser radiation, and especially waves characterized by relatively strong emission in any of the following frequency ranges, in 1/cm units, selected inter alia from a group consisting of about 3300 to about 3500; about 250 to about 3300; and about 150 to about 250.

It is also in the scope of the present invention to provide a method as defined above, comprising emitting ultrasound.

### BRIEF DESCRIPTION OF THE INVENTION

In order to understand the invention and to see how it may be implemented in practice, a preferred embodiment will now be described, by way of non-limiting example only, with reference to the accompanying drawing, in which

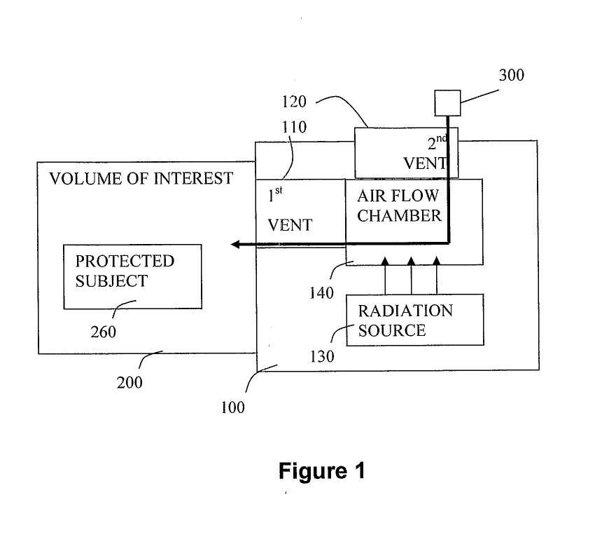

FIG. 1 schematically presents the present invention [100] employed to prevent infestation at a volume of interest [200], and comprising an fluid flow chamber [140];

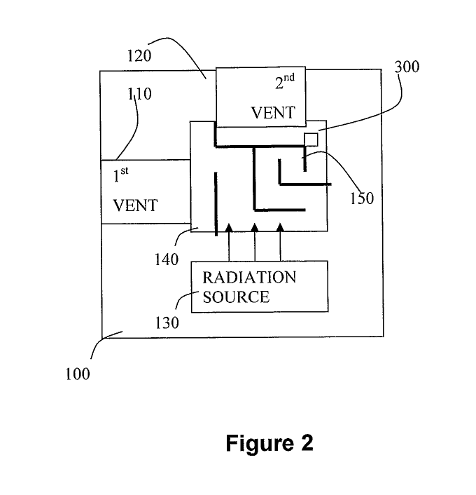

FIG. 2 schematically presents a baffle [150] at the fluid flow chamber [140];

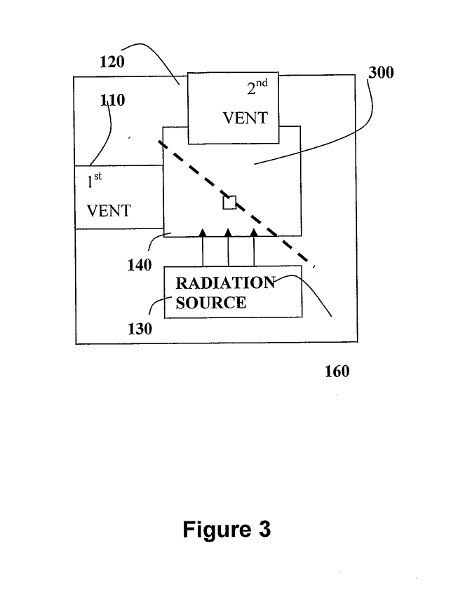

FIG. 3 schematically presents a filter or a bait [160] at the fluid flow chamber [140];

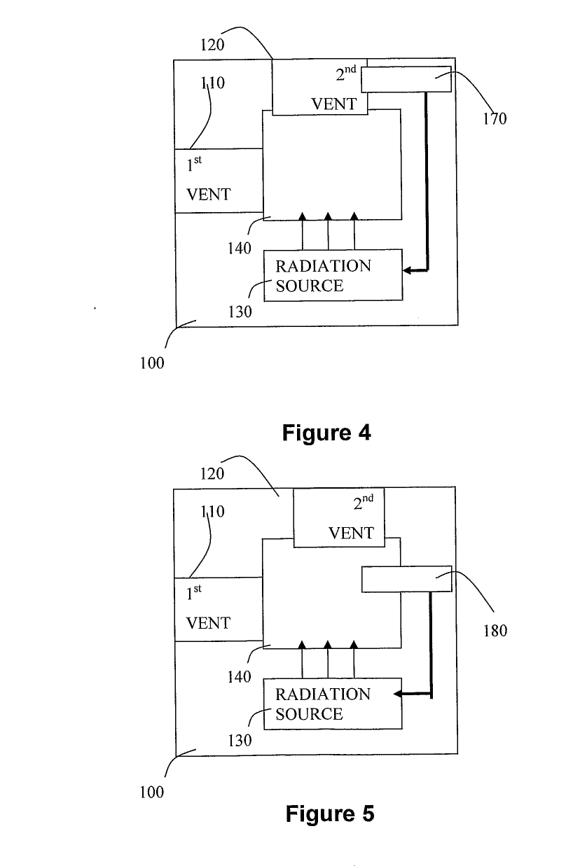

FIG. 4 schematically presents a detector [170] at a vent;

FIG. 5 schematically presents a detector [180] at the chamber;

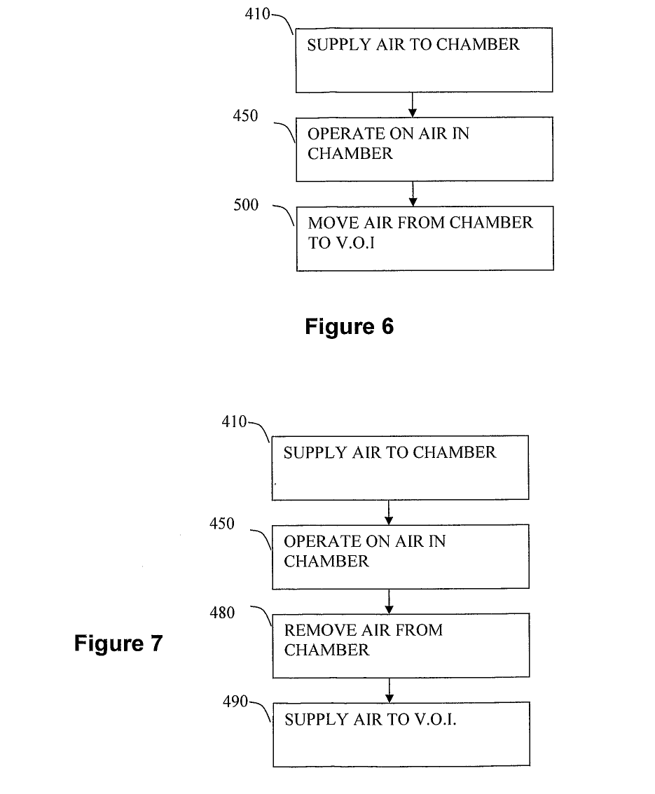

FIG. 6 schematically presents a pipeline of operations for the prevention of infestation in a volume of interest;

FIG. 7 schematically presents a pipeline of operations for the prevention of infestation, elaborating on the operation of moving fluid from the chamber to the volume of interest;

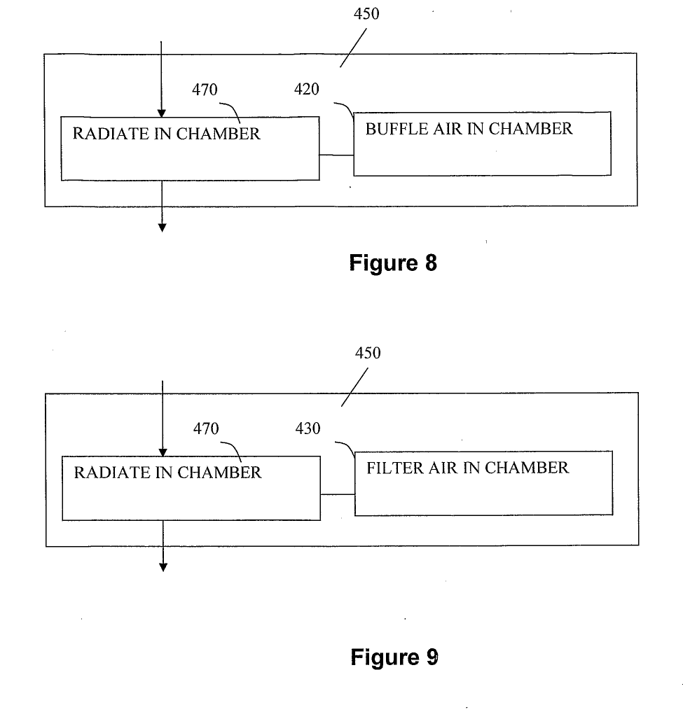

FIG. 8 schematically presents a pipeline operation including fluid baffling;

FIG. 9 schematically presents a pipeline operation including fluid filtering; and,

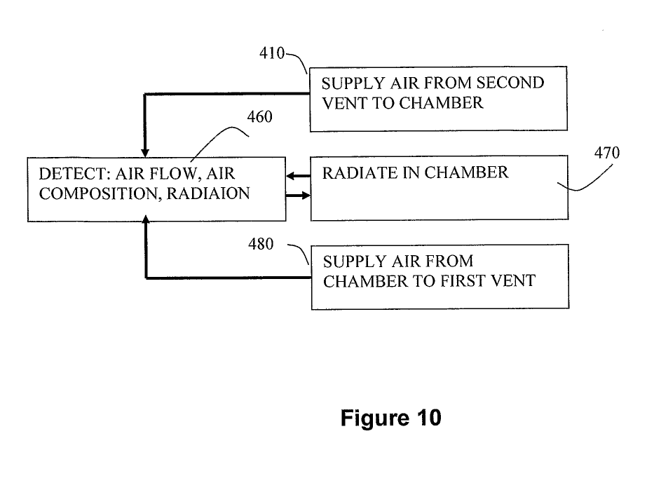

FIG. 10 schematically presents a method for the prevention of infestation employing feedback on radiation into a chamber;

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide apparatus and method for the prevention of infestation inside a volume of interest.

The term ‘infestation’ refers in the present invention to any infestation, infection or disease that is inflicted by one pathogen or more.

The term ‘pathogen’ refers in the present invention to any pathogenic organism or pest. Those pathogens or pests are selected in a non-limiting manner from insects, especially mosquitoes and other pests, particularly agricultural pests, and microorganisms, especially bacteria, such as Legionella pneumophila, Bacillus anthracis, Escherichia coli and the like viruses, molds, fungi, weedes etc. It is in the scope of the present invention wherein insects are selected in a non-limiting manner from Ants, Aphids, Armyworms, Bag worms, Bees, Beetles, Black vine weevils, Caterpillars, Chinch bugs, Cockroaches, Crickets, Earwings, Flies, Fungus gnats, Lacebugs, Grasshoppers, Japanese beetles, Lace-bugs, Leaf feeding caterpillars, Leaf hoppers, Leaf rollers Leaf miners, Mealybugs, Mites and especially Dust Mites, Mole crickets, Pantry pests, Pill bugs, Root weevils, Scales, Shore flies, Spider mites, Spotted tentaform leafminers, Thrips, Two-spotted mites, Wasps or Whiteflies.

The term ‘prevention’ in regarding infestation refers in the present invention to either preventing a certain infestation from happening, or the reduction of magnitude of infestation.

The term ‘volume of interest’ refers in the present invention to any volume inside which infestation is to be prevented.

The term ‘radiation’ refers in the present invention to the emission of waves such as electromagnetic waves or sonic waves, or the emission of particles such as in radioactive radiation. It is acknowledged in this respect that the term ‘radiation’ especially yet not exclusively relates to an effective dosage of radiation radiated and adapted to disable a pathogen.

The term ‘baffle’ refers in the present invention to the control or redirection of the flow of fluid, be it liquid or gas, and particularly to the control or redirection of the flow of fluid. A baffle is a baffler or an apparatus that baffles the flow of fluid.

The term ‘filter’ refers in the present invention to the separation of one substance from another, and particularly to the filtering of fluid to exclude solid particles or biological organisms, either dead or alive.

The terms ‘bait’ and ‘lure’ refer in the present invention to the attraction of a biological organism to propel itself into a certain location where the bait or lure is placed.

The term ‘about’ refers hereinafter to ±20% of the defined measure.

The apparatus for the prevention of infestation inside a volume of interest according to a most general embodiment of the present invention, is schematically characterized by a first vent supplying fluid to the volume of interest, a chamber supplying fluid to the first vent, a second vent supplying fluid to this chamber, and a radiation source supplying radiation to said chamber.

The chamber comprises a baffle, a filter or a bait or lure according to some specific embodiments of the apparatus.

Either the chamber or any of the vents comprises a fluid flow detector, a fluid composition detector, or a radiation detector, according to some specific embodiments of the apparatus.

The method for preventing of infestation inside a volume of interest according to a most general embodiment of the present invention, is schematically characterized either by a pipeline of operation comprising supplying fluid into the volume of interest through a vent, supplying fluid to the first vent through an fluid flow chamber, supplying fluid into the chamber through a second vent, and emitting radiation into the chamber, or by a pipeline of operations comprising moving fluid into the volume of interest from an fluid flow chamber, moving fluid into the chamber from outside the volume of interest, and emitting radiation into the chamber.

Any of the operations of supplying or moving fluid comprise detecting the properties of the fluid flow or the fluid composition in some specific embodiments of the method.

The operation of radiating the chamber comprises the detection or some radiation properties, the baffling of fluid flow, or the filtering of fluid in some specific embodiments of the method.

The radiation emitted into the camber comprises electromagnetic (such as microwaves), ultrasonic, or radioactive radiation according to some specific embodiments of both apparatus and method according to the present invention. Specific embodiments make use of specific regions of the electromagnetic spectrum.

Reference is thus made now to FIG. 1, presenting a schematic and generalized presentation of the aforementioned novel apparatus for the prevention of infestation inside a volume of interest in the context in which it is employed. The apparatus [100] prevents infestation in volume of interest [200]. The prevented infestation would be inflicted upon protected organism [260] residing inside the volume of interest by infesting organism [300] originating outside the volume of interest. The volume of interest is incompletely enclosed by walls, screens or partitions, which the infesting organism can not penetrate. Were the enclosure complete, then the infesting [300] would have been separated from the protected organism [260] and the infestation prevented. For example, the protected organism may be some cropping plants, such as strawberries, and the infecting organism my be some insect, in which case the volume of interest may comprise a hothouse surrounding the plants by glass of plastic walls from all sides except from below, and also comprising the ground on which the plants grow. Was the hothouse (and ground) completely enclosing the plants, plants would have been protected from infestation by the insects. Also for example, a fluid-tight enclosed room protects people living in it from infection by fluid-born organism such as fungi spores. The enclosure is often not complete, because there are openings installed in the walls defining the volume of interest through which fluid may enter or exit. Fluid can be made to exit the volume of interest in great speed or force, for example by narrowing the exit paths, or by using fluid pumps, and so it is possible to deter the entrance of the infesting organisms through the fluid exits. Description will thus now concentrate on the prevention of entrance of infesting organisms through the fluid entrances. There may be one or more fluid entrances to the volume of interest, as schematically depicted in FIG. 1, e.g., first vent [110]. This vent may comprise a pipe, a window or simply be an opening in the enclosure of the volume of interest. In the case that the vent comprises a pipe, it can be made of metal, plastic or any suitable material. FIG. 1 shows a situation in which the infecting organism may enter volume [200] only though first vent [110].

A chamber [140] is connected to the first vent, and also to a second vent [120]. Fluid may flow through this chamber into the volume of interest. The chamber may be a box made of plastic, metal, wood, or any other suitable material, and it needs not be made of the same materials forming the walls of the volume of interest. Fluid flows into the chamber through the second vent, which may comprise of a pipe, a chimney, a window or simply an opening in the chamber. In the case that the vent comprises a pipe or a chimney, it can be made of metal, plastic, bricks, clay or any suitable material. Infesting organism [300] comprises one or more organism belonging to one or more species, potentially able to infest, infect, prey upon, or other wise adversely effect the life cycle of the protected subject [260]. The infesting organism is able to pass with the fluid from through both vents and through the chamber into the volume of interest. The invention is most effective when the path described above is the only entrance path to the volume of interest.

Radiation source [130] is depicted in FIG. 1 as residing outside chamber [140]. This clarifies the description and highlights the functional distinction between these two elements of the present invention. However, the radiation source may as well be located inside the chamber, as long as it radiates into the chamber. Radiation source [130] radiates the volume in the chamber so that the radiation is intense inside the chamber. The radiation may comprise microwave radiation, in which case the source [130] comprises an emitter of microwave radiation. Especially, the emitter may be similar to that used in common microwave ovens, in which case the radiation is an electromagnetic wave of about 3400 [1/cm]. It is a well known fact that such radiation is efficient in heating water, and thus it has adverse effects on biological organism that contain water. If emitted in a sufficient dose, this radiation may kill infesting organisms passing through chamber [140]. Thus, the infecting organism may reach the volume of interest, but in a state preventing the infection. For example, the infecting organism may be dead when reaching the volume of interest. Further elaborating on the construction of the fluid flow chamber, it may be of benefit consider the type of radiation employed in the selection of the material of it construction. According to a specific embodiment of the present invention, the radiation comprises microwaves, and the chamber is made of metal sheets, to contain the radiation within the chamber, and achieve efficiency and safety, again in a fashion similar to that of microwave ovens. The following design considerations may be taken into account in the selection of the wavelength of the microwave, or more generally, of some electromagnetic radiation. Firstly, the price and availability of the source. Some sources are relatively cheap and available off-the-shelf because the find uses in other applications. Among them are microwave magnetron tubes, visible and infrared lamps and various types of lasers. Secondly, the consequences of leakage of radiation from the chamber. For example, leaking certain microwave and radio frequencies may disturb wireless communications in the region. Third, the wavelength may be tuned to achieve maximum effect on the infesting organisms, as it happens when it is close to some resonance wavelength of one of molecules present in the organism, be it water, any some organic molecule such as carbohydrates, proteins, fats, DNA, RNA or another.

For example, the following ranges of electromagnetic wave frequencies and known in the art to be efficiently absorbed by some organic molecules that key to organic life. The numbers are approximate and the units are 1/cm:

RNA or DNA about 200;

### Alkanes 2850-2960; 1350-1470; 675-1000;

Aromatic rings 675-870;

### Amines 3300-3500; 1180-1360;

Nitro compounds 1515-1560; 1345-1385.

It is hence in the scope of the invention wherein the disclosed systems and methods comprising means and steps, respectively, adapted for emitting radiation which is adapted to target the pathogen's ingredients, especially those that are selected in a non-limiting manner from a group consisting of RNA, DNA, alkanes, aromatic rings, amines and nitro compounds, or any combination thereof.

According to another embodiment of the present invention the radiation is provided by placing some highly radioactive material inside the chamber, in which case the chamber may be constructed of led for safety. According to yet another embodiment of the present invention, the radiation comprises ultrasonic waves and the chamber is best designed to achieve resonance of these waves inside the chamber, in accordance with the selected wavelength.

Reference is thus made now to FIG. 2, presenting a schematic and generalized presentation of an embodiment of aforementioned novel apparatus for the prevention of infestation inside a volume of interest (not shown), in which chamber [140], which is described in reference to FIG. 1 comprises a baffle [150]. The baffle, as it is defined herein above, controls or redirects the flow of fluid in the chamber, so that a particle carried in the flow of fluid may spend long time inside the chamber. This increases the time in which an infesting organism [300] spends in the chamber, and thus increases the amount of radiation energy it may absorb. According to a specific embodiment of the present invention the baffle comprises one or more plates made of metal, plastic, wood or any other suitable material, and according to another embodiment of the present invention the baffle comprises a pipe of metal, plastic, wood or any other suitable material, folded to fit the chamber and connecting the two vents. The selection of the material should preferably take the radiation emitted by the source [130] into account. According to one embodiment of the present invention the radiation comprises microwave, and the baffle avoid employing metal, but uses any material transparent to the waves, but according to another embodiment of the present invention the baffle does comprise metal plats or pipes positioned to act as a waveguide, with the radiation flowing in parallel to the fluid flow.

Reference is thus made now to FIG. 3, presenting a schematic and generalized presentation of an embodiment of aforementioned novel apparatus for the prevention of infestation inside a volume of interest (not shown), in which chamber [140], which is described in reference to FIG. 1 comprises a filter [160]. The filter may act a baffle as described in reference to FIG. 2 by slowing down the progress of the infesting organism through chamber [140], but it also block it from exiting the chamber altogether, either dead or alive. The filter may comprise a net or a sieve made by a suitable textile or plastic material, and constructed so that it does not interfere with the radiation, as explained in reference to FIG. 2, and does not completely block the flow of fluid. According to a specific embodiment of the present invention the filter may comprise a thin cotton cloth stretched across a wooden frame. According to another specific embodiment the present invention is fitted in conjunction with a pre-existing fluid condition system to avoid certain disease known to inflict certain buildings (such as the Legionaries disease), and the filter is similar to that used by the pre-existing system.

Reference is still made to FIG. 3, presenting a schematic and generalized presentation of an embodiment of aforementioned novel apparatus for the prevention of infestation inside a volume of interest, in which chamber [140] comprises bait or lure [160] acting to attract the infesting organism to enter the chamber. The bait may comprise of a source of light an emitter of some attractive scent. According to this embodiment of the present invention the chamber resides inside the volume of interest, in which case FIG. 1 is interpreted to schematically show the functional distinction between the apparatus according to the present invention [100], and the volume it protects [200].

Reference is thus made now to FIG. 4, presenting a schematic and generalized presentation of an embodiment of aforementioned novel apparatus for the prevention of infestation inside a volume of interest, in which one of the vents comprises a detector [170] detecting some properties of the fluid passing through the vent. FIG. 4 shows one embodiment of the present invention in which the detector detects some properties of the fluid at the second vent [120]. According to another embodiment of the present invention a similar detector detects similar properties at the first vent [110]. According to a specific embodiment of the present invention, the detector senses the amount of fluid flowing through the vent, and according to another embodiment of the present invention, the detector senses the composition of the fluid, especially the amount of infesting organisms present in the fluid. Detectors for sensing fluid flow are well known in the art. A specific implementation of such a apparatus may use an electrically heated wire which temperature is measured. This detector relies of the cooling properties of fluid flow. Detectors for sensing fluid composition are also well known in the art. A specific implementation of such a apparatus may pass a ray of light through the passing fluid and observe the scattering, reflection or absorption of light by the passing fluid composition.

Reference is thus made now to FIG. 5, presenting a schematic and generalized presentation of an embodiment of aforementioned novel apparatus for the prevention of infestation inside a volume of interest, in which the chamber [140] comprises a detector [180] detecting some properties the radiation present in the chamber. Apparatus for detecting the amplitude, wavelength or spectrum of radiation are well known in the art. According to a particular embodiment of the present invention the radiation comprises microwaves, and the detector comprises a microwave antenna.

Reference is thus made now to both FIGS. 4 and 5, schematically depicting embodiments according to the present invention that comprise various detectors. The detector may be couples to other components (not shown) such as amplifiers, attenuators, analog to digital converters, analog or digital processors, etc. The information obtained trough the detector [170] or [180] forms, according to such embodiments of the preset invention, a feedback loop controlling radiation source [130]. According to a specific embodiment of the present invention the radiation source is an emitter of microwaves, such as a magnetron tube. Controlling the output of such a apparatus is well known in the art. It is well known for microwave over, like those used as household appliances to control the output of radiation by varying the duty cycle of the operation of the source. Providing a feedback loop including a detector and the radiation source achieves several possible benefits. For example, it avoids waste by reducing the amount of radiation when the fluid flow is slow, or when no infesting organisms are present, it minimizes the amount of radiation that may leak outside the chamber, and it increases the effectiveness and efficiency of the present

Reference is thus made now to FIG. 6, presenting a schematic and generalized presentation of an embodiment of aforementioned novel method for the prevention of infestation inside a volume of interest. FIG. 6 depicts a pipeline comprising three operations. Normally all three operation can be performed simultaneously, but the subject of operations passes though them in a certain order as depicted, and as explained herein below. Vertical arrows depicted in this and the following figures schematically show the composition of various operations to form a pipeline. The first operation [410] comprises supplying fluid to a chamber from outside a volume of interest; the second operation [450] comprises radiating fluid in the chamber, and the third operation [500] comprises moving fluid from the chamber into the volume of interest. Further detail of the use and implementation of this pipeline are disclosed in reference to FIG. 1.

Reference is thus made now to FIG. 7, presenting a schematic and generalized presentation of an embodiment of aforementioned novel method for the prevention of infestation inside a volume of interest. FIG. 7 depicts a pipeline comprising four operations. Normally all four operation can be performed simultaneously, but the subject of operations passes though them in a certain order as depicted, and as explained herein below. The first operation [410] comprises supplying fluid to a chamber; the second operation [450] comprises radiating fluid in the chamber, the third operation [480] comprises removing fluid from the chamber; and the last operation [490] comprises supplying fluid to a volume of interest. Further detail of the use and implementation of this pipeline are disclosed in reference to FIG. 1.

Reference is thus made now to FIG. 8, presenting a schematic and generalized presentation of a detail of an embodiment of aforementioned novel method for the prevention of infestation inside a volume of interest. The detail shown in FIG. 8 elaborates on operation [450] shown in FIGS. 6 and 7. The connection by a horizontal line of two blocks in this figure schematically depicts the existence of two distinct simultaneous actions that act on the same subject. Thus, both the radiation of fluid in the chamber [470] and the baffling of fluid in the chamber [420] act of the same volume of fluid in the chamber at any given time, and together they form pipeline operation [450]. The act of baffling is described in detail in reference to FIG. 2.

Reference is thus made now to FIG. 9, presenting a schematic and generalized presentation of a detail of an embodiment of aforementioned novel method for the prevention of infestation inside a volume of interest. The detail shown in FIG. 9 elaborates on operation [450] shown in FIGS. 6 and 7. The connection by a horizontal line of two blocks in this figure schematically depicts the existence of two distinct simultaneous actions that act on the same subject. Thus, both the radiation of fluid in the chamber [470] and the filtering of fluid in the chamber [430] act of the same volume of fluid in the chamber at any given time, and together they form pipeline operation [450]. The act of filtering is described in detail in reference to FIG. 3.

Reference is thus made now to FIG. 10, presenting a schematic and generalized presentation of a detail of an embodiment of aforementioned novel method for the prevention of infestation inside a volume of interest. This figure shows some of the pipeline operations described in reference to the previous figures, but the vertical arrows showing the formation of a pipeline in previous figures are removed from this figure for clarity. The arrows in this figure schematically show a feedback loop controlling the operation of emitting radiation [470] that is described in reference to the previous figures. FIG. 10 shows the detection of some physical property [460], and the arrow leading from it to the operation of radiation symbolizes the influence of the act of detection on the act of radiation. Arrows leading to the act of detection [460] symbolize the relationship of the act of detection to other action according to some specific embodiments as follows. According to a specific embodiment of the present invention, detection is incorporated with the passage of fluid into the chamber [410] as described in reference to FIG. 4; according to another specific embodiment of the present invention, detection is incorporated with the passage of fluid from the chamber [480] as it is also explained in reference to FIG. 4; and according to yet another specific embodiment of the present invention, detection is incorporated with the action of radiating in the chamber [470], as explained in reference to FIG. 5.

The following is an example which illustrates a best mode for practicing the present invention. This example should not be construed as limiting. In this example the volume of interest is a greenhouse, a volume enclosed by a ceiling above, the earth below, and walls all around it. The walls and the ceiling are made of glass or polymeric panes supported by an aluminum framework. Strawberry plants are grown on the ground inside the volume of interest. Several windows are fixed in the walls through which air may enter the greenhouse. None of the windows are directly open to the outside, but all are connected to a chamber via plastic tubing. Small exits windows are fixed in the ceiling. Air heating and rising in the volume may exit through the exit windows, but one-directional valves prevent air from flowing in the opposite direction. The chamber is rectangular, is made of aluminum plates, and is placed on the ground. It has four walls facing four directions: north, south, east and west. The plastic tubing leading air to the greenhouse connect to the chamber through a window in its northern wall. Air enters the chamber through a window in its southern wall. A net is installed in the southern wall window. A source of microwave radiation is installed on the eastern wall, emitting radiation into the chamber. The source is similar to that found in microwave ovens, and the radiation's wavelength is about 3400 [1/cm]. The source of radiation is controlled by a personal computer fitted with control boards. The amount of radiation is constant when it is turned on, but its duty cycle is variable. A plastic propeller connected to a dynamo is placed at the northern window. The electrical current produced by the dynamo serves as a control signal to the microprocessor via an analog to digital converter in one of the control boards. The greater the flow of air through the chamber, the faster the propeller rotates, and the longer is the duty cycle of the radiation source. Plywood planks are installed in the chamber in parallel to the northern and southern walls, obstructing but not blocking the flow of air from south to north. The net on the southern window prevents the entrance of large insects into the greenhouse. Small insects are carried by the air flow into the chamber, but are killed by the microwave radiation. No living insects ever enter the greenhouse, and thus none can infect the strawberries.

Another mode is an automatic air-condition disinfecting system, e.g., central heating facility in a hospital, a food processing plant, medicaments producing laboratories etc, being a volume of interest to be continuously protected form fluid transferred pathogens, wherein outflow of heated ventilation air is enforced into a cascade of microwave emitters, some are tuned to emit about 3400 1/cm and others about 200 1/cm, so as bacteria (e.g., bacterium Legionella pneumophila) and other pathogens or pests (e.g., mosquitoes) are eliminated, and microorganism's contamination via hospital ventilation (e.g., Legionnaires' disease) is significantly reduced.

1. Apparatus [100] for the prevention of infestations inside a volume of interest (VOI) [200] caused by pathogens, said apparatus comprising:
a) a first vent [110] supplying a fluid into said VOI;
b) a chamber [140] supplying a fluid to said first vent;
c) a second vent [120] supplying a fluid into said chamber; and,
d) a radiation emitter [130] emitting radiation into said chamber hence disabling said pathogens.

2. The apparatus according to claim 1, wherein said chamber additionally comprising auxiliaries selected from a group including baffle, filter, lure or bait or combination thereof.

3. The apparatus according to claim 1, additionally comprising at least one detector selected from a group including composition detector, flow detector, radiation detector or any combination thereof.

4. The apparatus according to claim 1, wherein said radiation is an electromagnetic radiation.

5. The apparatus according to claim 4, wherein said radiation is microwave radiation, infrared radiation, ultraviolet radiation, visible light, gamma radiation, laser radiation, maser radiation or any combination thereof.

6. Apparatus according to claim 4, wherein said radiation is characterized by relatively strong emission in any of frequency ranges, in 1/cm units, selected from a group consisting of about 3300 to about 3500; about 250 to about 3300; and about 150 to about 250.

7. The apparatus according to claim 1, wherein said radiation comprising ultrasonic waves.

8. The apparatus according to claim 1, wherein said pathogen is selected from (i) insects, especially agricultural pests, and (ii) microorganisms, especially bacteria, viruses, molds, fungi, weedes and combination thereof.

9. A method of preventing of infestations inside a volume of interest caused by pathogens, said method comprising steps of
a) supplying fluid into said volume of interest through a first vent;
b) supplying fluid to said first vent through a chamber;
c) supplying fluid into said chamber through a second vent; and,
d) emitting an effective dosage of radiation into said chamber and hence disabling said pathogens.

10. The method according to claim 9, further comprising either baffling or filtering fluid passing through said chamber or both.

11. The method according to claim 9, further comprising attracting infesting organisms to enter into said chamber.

12. The method according to claim 9, further comprising controlling the emission of radiation into said chamber.

13. The method according to claim 12, further comprising detecting properties of radiation.

14. The method according to claim 12, comprising detecting fluid flow rate.

15. The method according to claim 12, comprising detecting fluid composition.

16. The method according to claim 9, wherein the action of emitting radiation comprising emitting electromagnetic waves.

17. The method according to claim 16, wherein the action of emitting radiation comprising emitting any radiation selected from a group including infrared radiation, ultraviolet radiation, visible light, gamma radiation, laser radiation, maser radiation or any combination thereof.

18. The method according to claim 16, wherein said step of emitting radiation comprising emitting relatively strong waves in any of frequency ranges, in 1/cm units, selected from a group consisting of about 3300 to about 3500, about 250 to about 3300, and about 150 to about 250.

19. The method according to claim 9, wherein said step of emitting radiation is adapted to target said pathogen's ingredients, selected from a group consisting of RNA, DNA, alkanes, aromatic rings, amines and nitro compounds, or any combination thereof.

20. The method according to claim 9, wherein remitting radiation into said chamber comprising emitting ultrasound.

21. The method for the prevention of infestations caused by pathogens inside a volume of interest, comprising steps of
a) flowing fluid from a chamber into said volume of interest;
b) flowing fluid into said chamber from outside of said volume of interest; and,
c) emitting an effective dosage of radiation into said chamber and hence disabling said pathogens.

22. The method according to claim 21, comprising either baffling or filtering fluid passing through said chamber or both.

23. The method according to claim 21, comprising controlling said emission of radiation into said chamber.

24. The method according to claim 23, comprising detecting properties of radiation.

25. The method according to claim 23, comprising detecting fluid flow rate.

26. The method according to claim 23, comprising detecting fluid composition.

27. The method according to claim 21, wherein emitting radiation comprising emitting electromagnetic waves.

28. The method according to claim 27, wherein emitting radiation into said chamber comprising step or steps of emitting radiation selected from a group including infrared radiation, ultraviolet radiation, visible light, gamma radiation, laser radiation, maser radiation or any combination thereof.

29. The method according to claim 27, wherein said emitting radiation comprises step or steps of emitting relatively strong waves in any of frequency ranges, in 1/cm units selected from a group consisting of about 3300 to about 3500, about 250 to about 3300, and about 150 to about 250.

30. The method according to claim 21, wherein remitting radiation into said chamber comprising emitting ultrasound.
